(19) United States
(12) Patent Application Publication
(10) Pub. No.: US 2023/0348586 A1
(43) Pub. Date: Nov. 2, 2023

(54) THERAPEUTIC AGENTS AND USES THEREOF

(71) Applicant: THE BOARD OF REGENTS OF THE UNIVERSITY OF TEXAS SYSTEM, Austin, TX (US)

(72) Inventors: Phillipp E. SCHERER, Southlake, TX (US); Shangang ZHAO, Dallas, TX (US); Zhiqiang AN, Austin, TX (US); Ningyan ZHANG, Austin, TX (US)

(73) Assignee: THE BOARD OF REGENTS OF THE UNIVERSITY OF TEXAS SYSTEM, Austin, TX (US)

(21) Appl. No.: 18/026,696

(22) PCT Filed: Sep. 21, 2021
(86) PCT No.: PCT/US2021/051304

§ 371 Date: Mar. 16, 2023

Related U.S. Application Data
(60) Provisional application No. 63/080,910, filed on Sep. 21, 2020.

(51) Int. Cl. C07K 16/26; A61K 38/46; A61K 31/7088; A61K 38/22; C12N 15/113; A61K 38/26; A61K 39/395; A61P 3/04; A61P 35/02; A61P 35/00; A61P 9/00; A61P 1/16

(52) U.S. Cl. CPC C07K 16/26; A61K 38/465; A61K 31/7088; A61K 38/2264; C12N 15/1136; A61K 38/26; A61K 39/3955; A61P 3/04; A61P 35/02; A61P 35/00; A61P 9/00; A61P 1/16; C12N 2310/14; C12N 2310/531; A61K 2039/505

(57) ABSTRACT

The present disclosure relates generally to therapeutic agents and related uses thereof, including, agents for reducing leptin in a patient or subject and methods of treatment thereof. The therapeutic agents can comprise (without limitation), an antibody or specific binding fragment thereof, a leptin antagonist, a leptin targeting antisense oligonucleotide, a leptin targeting small interfering RNA (siRNA), a leptin targeting short hairpin RNA (shRNA), and/or a gene editing composition directed to at least one target sequence of a leptin polynucleotide. The therapeutic agents can be used in various methods of treatment, including (without limitation), treating liver fibrosis, cancer, inducing or maintaining weight loss, reducing or preventing weight gain, and increasing insulin sensitivity, among others.

### PRIORITY

This application claims the benefit of U.S. Ser. No. 63/080,901, filed on Sep. 21, 2021, and incorporated herein in its entirety.

### TECHNICAL FIELD

The present disclosure relates generally to therapeutic agents and related uses thereof, including, agents for reducing circulating leptin in a patient or subject and methods of treatment thereof.

### BACKGROUND

Leptin is a hormone predominantly released by adipose cells and plays a role in the regulation of fat storage. Therapeutic agents, for example, therapeutic agents that reduce leptin levels, are needed in the art for the treatment of various diseases or conditions.

### SUMMARY

The present disclosure relates to methods of treatment. In some embodiments, methods of treating liver disease are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of treating liver fibrosis are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of treating liver cirrhosis are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of maintaining weight loss are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of treating cancer are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of treating colorectal cancer are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of treating acute lymphoblastic leukemia are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of treating cardiovascular disease or one or more symptoms of cardiovascular disease are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of reducing fasting glycemia are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of improving glucose tolerance are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of reducing an amount of GLP-1 agonist delivered to a subject are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of increasing insulin sensitivity within 24 or fewer hours are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of reducing inflammation and fibrosis in COVID-19 infections are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of inducing breast cancer regression are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of enhancing effectiveness of PD-1 checkpoint inhibitors are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of providing metabolic improvements for ciliopathy or Bardet-Biedel Syndrome are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, methods of providing metabolic improvements for polycystic ovary syndrome (PCOS) are provided. The methods comprise administering a therapeutic agent for lowering circulating leptin to a subject in need thereof, wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

In some embodiments, a method of inducing weight loss in a patient in need thereof is provided. The method comprises administering a treatment regimen comprising a therapeutic agent for lowering circulating leptin and a GLP-1 agonist to a subject in need thereof, wherein the GLP-1 agonist is liraglutide, exenatide, albiglutide, dulaglutide, lixisenatide, or semaglutide. The method can further comprise removing the GLP-1 agonist from the treatment regimen after a desired weight level is achieved.

In some embodiments a method of reducing weight gain resulting from administration of an anti-psychotic medication is provided. The method comprises administering an anti-psychotic medication and a therapeutic agent for lowering circulating leptin to a subject in need thereof.

In some embodiments, the antibody is hLept-1, hLept-2, hLept-3, hLept-4, hLept-5, or hLept-6, and the specific binding fragment is obtained from hLept-1, hLept-2, hLept-3, hLept-4, hLept-5, or hLept-6. The antibody or specific binding fragment can have a variable heavy chain (V_{H}) CDR1 sequence as set forth in SEQ ID NOs: 1, 2, 3, 4 or 5; a V_{H} CDR2 sequence as set forth in SEQ ID NOs: 6, 7, 8, 9 or 10; a V_{H} CDR3 sequence as set forth in SEQ ID NOs: 11, 12, 13, 14 or 15; a variable light chain (V_{L}) CDR1 sequence as set forth in SEQ ID NOs: 16, 17, 18, 19 or 20; a V_{L} CDR2 sequence as set forth in SEQ ID NOs: 21, 22, 23, 24 or 25; and a V_{L} CDR3 sequence as set forth in SEQ ID NOs: 26, 27, 28, 29 or 30.

In some embodiments, the gene editing composition can comprise at least one polynucleotide encoding an RNA-guided DNA endonuclease protein or an RNA-guided DNA endonuclease protein, and at least one guide RNA (gRNA) having a spacer sequence complementary to a leptin polynucleotide sequence.

In some embodiments, the leptin antagonist is a leptin mutein. The leptin mutein can be LanI (L39A/D40A/F41A mutant), Lan2 (L39A/D40A/F41A/I42A mutant), or SHLA (D23L/L39 A/D40A/F41A mutant.

In some embodiments the amount of circulating leptin is lowered by 30 to 90% in the subject.

### BRIEF DESCRIPTION OF DRAWINGS

The foregoing summary and the following detailed description are better understood when read in conjunction with the appended drawings. Exemplary embodiments are shown in the drawings; however, it is understood that the embodiments are not limited to the specific structures depicted herein. In the drawings:

FIG. 1A shows glycemia levels (mg/dl) over time before leptin antibody treatment, according to Example 1 and exemplary embodiments of the present disclosure.

FIG. 1B shows glycemia levels (mg/dl) over time after leptin antibody treatment, according to Example 1 and exemplary embodiments of the present disclosure.

FIG. 1C shows glucose infusion rate (GIR) before and after leptin antibody treatment, according to Example 1 and exemplary embodiments of the present disclosure.

FIG. 2A shows expression of Col1a1 gene in livers after leptin antibody treatment, according to Example 2 and exemplary embodiments of the present disclosure.

FIG. 2B shows expression of Col3a1 gene in livers after leptin antibody treatment, according to Example 2 and exemplary embodiments of the present disclosure.

FIG. 2C shows expression of Col4a4 gene in livers after leptin antibody treatment, according to Example 2 and exemplary embodiments of the present disclosure.

FIG. 2D shows expression of transforming growth factor beta (TGF-β) in livers after leptin antibody treatment, according to Example 2 and exemplary embodiments of the present disclosure.

FIG. 3A shows body weight (g) over time during leptin antibody or leptin antibody+GLP-1 agonist treatment, according to Example 3 and exemplary embodiments of the present disclosure.

FIG. 3B shows body weight gain (g) over time during leptin antibody or leptin antibody+GLP-1 agonist treatment, according to Example 3 and exemplary embodiments of the present disclosure.

FIG. 3C shows body weight change (percentage) over time during leptin antibody or leptin antibody+GLP-1 agonist treatment, according to Example 3 and exemplary embodiments of the present disclosure.

FIG. 4A shows circulating leptin level (ng/ml) over time during treatment of liraglutide and after removal of liraglutide, according to Example 4 and exemplary embodiments of the present disclosure.

FIG. 4B shows body weight (g) over time during leptin antibody treatment during liraglutide treatment and liraglutide withdrawal, according to Example 4 and exemplary embodiments of the present disclosure.

FIG. 4C shows body weight gain (g) over time during leptin antibody treatment, during liraglutide treatment and liraglutide withdrawal, according to Example 4 and exemplary embodiments of the present disclosure.

FIG. 4D shows glycemia levels (mg/dl) over time during leptin antibody treatment based on oral glucose tolerance test (OGTT), according to Example 4 and exemplary embodiments of the present disclosure.

FIG. 5A shows body weight (g) over time during olanzapine and leptin antibody treatment, according to Example 5 and exemplary embodiments of the present disclosure.

FIG. 5B shows daily food intake (g) during olanzapine and leptin antibody treatment, according to Example 5 and exemplary embodiments of the present disclosure.

FIG. 6 shows tumor volume (mm³) over time after leptin antibody treatment according to Example 6 and exemplary embodiments of the present disclosure.

FIG. 7A shows Green Fluorescent Protein (GFP) expression over time after fasting treatment group (C: fasting), leptin antibody treatment group (B: Anti-Lep) and control group (A: IgG), according to Example 7 and exemplary embodiments of the present disclosure.

FIG. 7B shows percent survival of N-Myc proto-oncogene protein over time after fasting treatment group (C: fasting), leptin antibody treatment group (B: Anti-Lep) and control group (A: IgG), according to Example 7 and exemplary embodiments of the present disclosure.

FIGS. 8A to 8E generally show regulation of leptin expression under multiple physiological stimuli. The data in FIGS. 8A to 8E are given as mean±SEM and the error bars indicated SEM. FIG. 8A shows expression levels of leptin in various fat depots, according to Example 8 and exemplary embodiments of the present disclosure.

FIG. 8B shows expression of leptin under short periods of cold exposure, according to Example 8 and exemplary embodiments of the present disclosure.

FIG. 8C shows expression of leptin under thermal neutral conditions, according to Example 8 and exemplary embodiments of the present disclosure.

FIG. 8D shows expression of leptin under acute high fat diet (HFD), according to Example 8 and exemplary embodiments of the present disclosure.

FIG. 8E shows circulating leptin level under acute HFD, according to Example 8 and exemplary embodiments of the present disclosure.

FIG. 9 shows the effects of LepAB on MC38-associated tumor growth in mice, according to Example 9 and exemplary embodiments of the present disclosure.

### DETAILED DESCRIPTION

The terminology used in the present disclosure is for the purpose of describing particular exemplary embodiments and is not intended to be limiting. As used in the description of the embodiments of the disclosure and the appended claims, the singular forms “a,” “an,” and “the” are intended to include the plural forms as well, unless the context clearly indicates otherwise.

The term “and/or,” as used herein, refers to and encompasses any and all possible combinations of one or more of the associated listed items.

The term “about,” as used herein when referring to a measurable value such as an amount of a component, time, temperature, and the like, is meant to encompass variations of 5%, 1%, 0.5%, or even 0.1% of the specified amount. Unless otherwise defined, all terms, including technical and scientific terms used in the description, have the same meaning as commonly understood by one of ordinary skill in the art to which this disclosure belongs.

A “patient” or “subject” as used herein is a mammal, e.g., a human or a veterinary patient or subject, e.g., mouse, rat, guinea pig, dog, cat, horse, cow, pig, or non-human primate, such as a monkey, chimpanzee, baboon or gorilla.

The term “treating” or “treatment” is meant to encompass administering to a subject agent(s) of the present disclosure for the purposes of amelioration of one or more symptoms of a disease or disorder, including palliative care. A “therapeutically effective amount” refers to the minimum amount of the active agent which effects treatment.

Embodiments of the present disclosure include treating various patient populations, diseases and conditions by regulating circulating levels of leptin. Leptin is a 167 amino acid product of a human leptin gene (UniProtKB A4D0Y8; P41159). Circulating leptin levels reflect the amount of energy stores in adipose tissue: higher leptin levels correlate to higher Body Mass Index (BMI) and higher fatty mass. Circulating leptin levels can also reflect acute changes in caloric intake as overfeeding tends to increase leptin secretion and fasting tends to reduce leptin secretion. Leptin levels direct the central nervous system in regulating energy homeostasis, neuroendocrine function, and metabolism. Circulating leptin levels in humans with a healthy BMI (i.e., 18.5-24.9) can be about 5.0, 7.5, or 10 ng/mL in blood or serum. Circulating leptin levels in humans with an overweight BMI (i.e., abut 25.0-29.9) or obese BMI (above 30) can be about 15, 20, 25, 30, 35, 40 or more ng/mL in blood or serum.

Circulating leptin can be measured using, for example, a radio-immunoassay, coated tube immune-radiometric, enzyme-linked immunosorbent assay (ELISA), or any other suitable assay. Circulating leptin (i.e., leptin that can move through the bloodstream) is present in blood or serum and is not associated or bound to cells or non-circulating receptors, Circulating leptin can be bound to a soluble receptor or other circulating macromolecule, for example, a soluble form of a leptin receptor and still be considered “circulating.” Soluble leptin receptor (sOB-R) circulates in two different N-glycosylated isoforms, as a dimer or in an oligomerized state. Leptin is not considered “circulating” where it is bound to leptin receptors in or on cells or tissues. Leptin can bind to specific leptin receptors (ObRs) in the brain and in peripheral tissues. Leptin binding works though several signal transduction pathways: leptin can bind to the Janus Kinase-Signal Transducer and Activator of Transcription-3 (JAK-STAT3), which pathway helps regulate energy homeostasis; Leptin works through the Phosphatidylinositol 2-Kinase (PI3K) pathway to regulate food intake and glucose homeostasis. Regulating levels of leptin can mean reducing or lowering circulating leptin in a patient or subject. In some embodiments, methods described herein can reduce circulating leptin levels by about 2, 5, 7, 10, 15, 20, 25, 30 or more ng/mL in blood or serum. In some embodiments, methods described herein can reduce circulating leptin levels by about 1, 2, 5, 7, 10, 15, 20, 30, 40, 50, 60 70% or more in blood or serum.

Patient populations can be any population of patients or subjects that have various diseases or conditions and/or is in need of a reduced or lowered circulating leptin level, for example (without limitation), patients or subjects that have liver fibrosis, obesity, cancer such as colorectal cancer, and/or insulin sensitivity or that have a need to reduce weight gain or to maintain or induce weight loss.

Therapeutic Compositions

Antibodies and Specific Binding Fragments

Therapeutic agents of the present disclosure can include various antibodies or specific binding fragment thereof. The term “antibody” as used herein is used broadly and can encompass polyclonal antibodies, monoclonal antibodies as well as specific binding fragments thereof. An antibody molecule can be monospecific, idiospecific, heterospecific, or polyspecific. Antibody molecules can have specific binding sites that bind to specific antigenic determinants, epitopes, on antigens. “Specific binding fragments” can comprise a portion of the full-length antibody. The portion can generally be the antigen binding or a variable region of the antibody. Examples of antibody fragments include Fab, Fab′, F(ab′)2 and Fv fragments; diabodies; linear antibodies; single-chain antibody molecules; and multispecific antibodies formed from antibody fragments.

In an embodiment, an antibody is an anti-leptin antibody or specific binding fragment thereof that specifically binds circulating human leptin. The antibodies can be one or more of hLept-1, hLept-2, hLept-3, hLept-4, hLept-5, and/or hLept-6, for example as described in PCT Publication No. WO2019/241660, which is incorporated herein by reference. Any other suitable leptin specific antibody can be used, e.g., those described in Mahmoudian et al., Hybridoma (Larchmt) 31:372 (2012), leptin monoclonal antibody 44802 (Invitrogen), monoclonal antibody 398 (R&D Systems), monoclonal antibody 3G7 (BioRad Antibodies). One or more specific binding fragments can be obtained from hLept-1, hLept-2, hLept-3, hLept-4, hLept-5, hLept-6, and or any other suitable leptin specific antibody. One or more of the binding fragments, for example, hLept-3, can be been crystallized with leptin bound. The antibodies or specific binding fragments can have a variable heavy chain (V_{H}) CDR1 sequence as set forth in SEQ ID NOs: 1, 2, 3, 4 or 5, a V_{H} CDR2 sequence as set forth in SEQ ID NOs: 6, 7, 8, 9 or 10, and/or a V_{H} CDR3 sequence as set forth in SEQ ID NOs: 11, 12, 13, 14 or 15. The antibodies or specific binding fragments can have a variable light chain (V_{L}) CDR1 sequence as set forth in SEQ ID NOs: 16, 17, 28, 19 or 20, a V_{L} CDR2 sequence as set forth in SEQ ID NOs: 21, 22, 23, 24 or 25, and/or a V_{L} CDR3 sequence as set forth in SEQ ID NOs: 26, 27, 28, 29 or 30. The antibodies or specific binding fragments can be dosed at about 0.1 to about 50 mg/kg, for example, about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45 or 50 mg·kg. In an embodiment, the antibodies or specific binding fragments can be dosed at about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg.

TABLE 1

| V_{H} | CDR1 | CDR2 | CDR3 |
|---|---|---|---|
| hLept-1V_{H} | SEQ ID NO: 1 | SEQ ID NO: 6 | SEQ ID NO: 11 |
| | GGSVSRGSHY | IHTDGST | AREPGGALNF |
| hLept-2V_{H} | SEQ ID NO: 2 | SEQ ID NO: 7 | SEQ ID NO: 12 |
| | GYTFTGYY | INPNSGGT | ASGKTYYDFWSGGRRGMDV |
| hLept-3V_{H} | SEQ ID NO: 3 | SEQ ID NO: 8 | SEQ ID NO: 13 |
| | GGTFSSYA | IIPIFGTA | ARSQVPSSYYYGMDV |
| hLept-5V_{H} | SEQ ID NO: 4 | SEQ ID NO: 9 | SEQ ID NO: 14 |
| | GFTFSSYA | ISYDGSNK | ARGREYYYYMDV |
| hLept-6V_{H} | SEQ ID NO: 5 | SEQ ID NO: 10 | SEQ ID NO: 15 |
| | GYTFTSYY | INPSGGST | ARGFGYGGKALDY |

TABLE 2

| V_{L} | CDR1 | CDR2 | CDR3 |
|---|---|---|---|
| hLept-1V_{L} | SEQ ID NO: 16 | SEQ ID NO: 21 | SEQ ID NO: 26 |
| | SSNIGSNT | SNN | ASWDDSLNGVV |
| hLept-2V_{L} | SEQ ID NO: 17 | SEQ ID NO: 22 | SEQ ID NO: 27 |
| | QSVSRY | TSS | QQTYSTPWT |
| hLept-3V_{L} | SEQ ID NO: 18 | SEQ ID NO: 23 | SEQ ID NO: 28 |
| | NSNIGAGYH | GDT | QSYDRSRGGWF |
| hLept-5V_{L} | SEQ ID NO: 19 | SEQ ID NO: 24 | SEQ ID NO: 29 |
| | NIARKS | NDN | QVWDNSDYV |
| hLept-6V_{L} | SEQ ID NO: 20 | SEQ ID NO: 25 | SEQ ID NO: 30 |
| | QNINSR | KAS | QQFDKYSIT |

Leptin Antagonists

Therapeutic agents of the present disclosure can include one or more leptin antagonists. The one or more leptin antagonist can be a leptin mutein. The leptin mutein can be one or more of LanI (L39A/D40A/F41A mutant), Lan2 (L39A/D40A/F41A/I42A mutant), or SHLA (D23L/L39 A/D40A/F41A mutant).

Antisense Oligonucleotides, RNAi, siRNA Molecules, and shRNA Molecules

Therapeutic agents of the present disclosure can include one or more leptin targeting antisense oligonucleotides. Antisense oligonucleotides are short, synthetic, single-stranded oligodeoxynucleotides that are complementary to the mRNA target. Antisense oligonucleotides can be about 20 nucleotides long and can be selected to target either the methionine (AUG) initiation codon, blocking translation, or the splice sites, to block splicing. Antisense oligonucleotides can be synthesized using chemically modified nucleotides, for example (without limitation), phosphorothioates, 2′-O-methyl RNA, or locked nucleic acids, which can confer nuclease resistance. Antisense oligonucleotides can hybridize to target RNA in a sequence-specific manner. Antisense oligonucleotides can inhibit gene expression, modulate splicing of a precursor mRNA, or inactivate microRNA. Antisense oligonucleotides can work by inducing RNase H endonuclease activity that cleaves the RNA-DNA heteroduplex and thereby can reduce target gene translation, for example, LEP, the gene encoding the hormone leptin. Antisense oligonucleotides can also inhibit 5′ cap formation, alter the splicing process (splice-switching), and sterically hinder ribosomal activity). In an embodiment, the leptin targeting antisense oligonucleotide can be administered in an amount of about 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 100, 135, 150, 175, 200, 225, 250, 275, 300, 325, 350, 375, 400, 425, 450, 475, 500, 550, 600, 650, 700, 750, 800, 850, 900, 950, or 1000 mg/kg.

RNAi

RNAi is a conserved biological response to double-stranded RNA that mediates resistance to both endogenous parasitic and exogenous pathogenic nucleic acids and regulates the expression of protein-coding genes. RNAi interrogates gene function by blocking gene expression and analyzing its effect on phenotype. RNAi silences genes by generating knockdowns at the mRNA level.

siRNA

Therapeutic agents of the present disclosure can include one or more small interfering RNA (siRNA) molecules targeting leptin. siRNA, also referred to as short interfering RNA or silencing RNA, is a class of double-stranded RNA non-coding molecules, which can be about 20-27 base pairs long. siRNA can operate within the RNA interference (RNAi) pathway. An endoribonuclease, Dicer, can cleave long dsRNA forming siRNA. Long dsRNA can come from hairpin, complementary RNAs, and/or RNA-dependent RNA polymerases. An siRNA can be transfected into a host cell, optionally within a vector such as a viral or non-viral vector. Once siRNA enters the target cell, proteins can come together to form the RNA-Induced Silencing Complex (RISC). After RISC forms, the siRNA can unwind to form two single stranded siRNA segments, the passenger strand and the guide strand. The passenger strand is degraded while the less thermodynamically stable guide strand remains part of the RISC and scans to find complementary mRNA. When the siRNA (part of RISC) binds to target mRNA, it induces mRNA cleavage. The cut mRNA is identified as abnormal by the cell and is degraded, thereby preventing translation and silencing the gene that encodes that mRNA, for example, a leptin gene.

siRNA can be used, for example, to decrease or downregulate gene expression of leptin, for example human leptin or other mammalian leptin. For example, siRNA can inhibit protein expression of leptin.

siRNA molecules can be about 20-27 nucleotides in length. In some embodiments, the first nucleotide of an siRNA can begin at nucleotide position 599, 673, 327, 241, 704, 672, 1016, 570, 670, 361, 694, 572, or 234 of GenBank Accession number NM_000230 (human leptin mRNA) and extend to about 20, 21, 22, 23, 24, 25, 27, or more nucleotides in length. An siRNA molecule can comprise one or more chemical modifications or other modifications. In an embodiment, the siRNA can be administered to a patient in an amount of about 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 100, 135, 150, 175, 200, 225, 250, 275, 300, 325, 350, 375, 400, 425, 450, 475, 500, 550, 600, 650, 700, 750, 800, 850, 900, 950, or 1000 mg/kg.

shRNA

Therapeutic agents of the present disclosure can include one or more short hairpin RNA (shRNA) targeting leptin, for example human leptin or mammalian leptin. shRNA, also referred to as small hairpin RNA or Hairpin Vector, is an artificial RNA molecule with a tight hairpin turn that can be used to silence target gene expression operating within the RNAi pathway. As discussed above, one method for gene knockdown can be transfection of exogenous siRNA, but transfected siRNA can degrade. An expression vector encoding an shRNA can be delivered to a subject. The expression vector can be viral or non-viral DNA vectors that encode shRNA. A common vehicle for shRNA delivery is viral transduction. Expression through AAV or adenovirus can prevent insertional mutagenesis since these vectors remain episomal. Expression through lentivirus provides a stable solution through chromosomal integration. The siRNA sequence can be modified to contain a short loop between the two strands, creating the shRNA. Dicer can process shRNA, which can provide an advantage over transfected siRNA, which can degrade more rapidly than shRNA. The one or more shRNAs targeting leptin can be, for example, one or more of:

(SEQ ID NO: 31)
CCTTCCAGAAACGTGATCCAA
(SEQ ID NO: 32)
GTCACCGGTTTGGACTTCATT
(SEQ ID NO: 33)
CATCCTGACCTTATCCAAGAT

In an embodiment, the shRNA can be administered to a patient in an amount of about 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 100, 135, 150, 175, 200, 225, 250, 275, 300, 325, 350, 375, 400, 425, 450, 475, 500, 550, 600, 650, 700, 750, 800, 850, 900, 950, or 1000 mg/kg.

In an embodiment, the one or more leptin targeting shRNAs can be, for example:

Human setting:
In 3′UTR:
shLEP1, entrezID_3952_2498_v2,
(SEQ ID NO: 34)
TTTAAATTCTCAGTTATCTTGT
potential off-targets can be: ITPR2, WIPF3 (predicted: CNTLN)
shLEP2, entrezID_3952_2497_v2,
(SEQ ID NO: 35)
TTAAATTCTCAGTTATCTTGTT
potential off-targets can be: ITPR2, WIPF3 (CNTLN)
shLEP3, entrezID_3952_2841_v2,
(SEQ ID NO: 36)
TTAATATCAAACTTCTTTACCC
potential off-targets can be: PLS1
shLEP4, entrezID_3952_1432_v2,
(SEQ ID NO: 37)
TTATTCAGAAAACACATTCTAG
potential off-targets can be: none
Mouse setting:
In 3UTR:
shLep11, entrezID_16846_2402_v2,
(SEQ ID NO: 38)
TATATATACTCAAATATACCTA
potential off-targets can be: none
shLep12, entrezID_16846_2455_v2,
(SEQ ID NO: 39)
TATAAATGAACTTCATGTTTAT
potential off-targets can be: none (pred.: Gm41257)
shLep13, entrezID_16846_3201_v2,
(SEQ ID NO: 40)
TAAAACAAAATTTTGTTGTTGC
potential off-targets can be: 4930470G03Rik,
6030458C11Rik (pred.: Slc35a3,)
shLep14, entrezID_16846_2462_v2,
(SEQ ID NO: 41)
TATGAAATATAAATGAACTTCA
potential off-targets can be: none (pred.: Gm34249, Mup-ps12, Pard3b,
Gm41257)
shLep15, entrezID_16846_2225_v2,
(SEQ ID NO: 42)
TATGTAAATGCAATAGACTGCA
potential off-targets can be: none
In coding sequence:
shLep16, entrezID_16846_178_v2,
(SEQ ID NO: 43)
TGTGAAATGTCATTGATCCTGG
potential off-targets can be: none
shLep17, entrezID_16846_176_v2,
(SEQ ID NO: 44)
TGAAATGTCATTGATCCTGGTG
shLep18, entrezID_16846_98_v2,
(SEQ ID NO: 45)
TTGAACATAAGACAGATAGGAC
shLep19, entrezID_16846_520_v2,
(SEQ ID NO: 46)
AACTGTTGAAGAATGTCCTGCA
shLep20, entrezID_16846_259_v2,
(SEQ ID NO: 47)
TTGGACAAACTCAGAATGGGGT

CRISPR Systems and Gene Editing Compositions

Therapeutic agents can include one or more gene-editing compositions directed to target at least one sequence of a leptin polynucleotide. The one or more gene editing compositions can comprise at least one polynucleotide encoding an RNA-guided DNA endonuclease protein, and at least one guide RNA (gRNA) having a spacer sequence complementary to a leptin polynucleotide sequence. These RNA-guided DNA endonucleases are directed by gRNA to cleave phosphodiester bonds within a polynucleotide chain. These gRNAs can be noncoding short RNA sequences that bind to complementary DNA sequences and can be used in DNA editing. One RNA-guided DNA endonuclease is CRISPR associated protein 9 (Cas9), which can cleave nearly any sequence complementary to the gRNA. However, any suitable RNA-guided DNA endonuclease can be used (e.g., Cas1, Cas1 B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a homolog, codon-optimized, or modified version thereof). The gRNA can confer target sequence specificity to the CRISPR-CAS9 system (or other suitable system) by first binding to the RNA-guided DNA endonuclease. Then, the gRNA sequence can guide the complex to a specific location on the DNA where RNA-guided DNA endonuclease performs its endonuclease activity cutting the target DNA strand.

CRISPR interrogates gene function by blocking gene expression and analyzing its effect on phenotype. CRISPR generates knockouts at the DNA level. CRISPR-based genome editing requires two components: a guide RNA and a CRISPR-associated endonuclease protein (Cas). The guide RNA, analogous to a GPS system, directs the Cas nuclease to the specific target DNA sequence, which then cuts the DNA at that site. The most commonly used nuclease, SpCas9, is the one isolated from the bacterium Streptococcus pyogenes, however, any suitable RNA-guided DNA endonuclease can be used.

Therapeutic agents can include one or more gene-editing compositions directed to at least one target sequence of a leptin polynucleotide (NCBI Gene ID: 3952). The one or more gene editing compositions can comprise at least one polynucleotide encoding an RNA-guided DNA endonuclease protein, and at least one guide RNA (gRNA) having a spacer sequence complementary to a leptin polynucleotide sequence. These RNA-guided DNA endonucleases are directed by gRNA to cleave phosphodiester bonds within a polynucleotide chain. These gRNAs can be noncoding short RNA sequences that bind to complementary DNA sequences and can be used in DNA editing.

In some embodiments, a guide RNA may comprise two RNA molecules, a first RNA molecule comprising a CRISPR-RNA (crRNA), and a second RNA molecule comprising a transactivating crRNA (tracrRNA). The first and second RNA molecules can form a RNA duplex via the base pairing between the hairpin on the crRNA and the tracrRNA. The crRNA contains an RNA sequence complementary to the selected target nucleic acid sequence (i.e., leptin). The tracrRNA acts as a bridge between the CRISPR-Cas protein (e.g., Cas 9). In other embodiments, the guide RNA can comprise a single RNA molecule and is known as a “single guide RNA” or “sgRNA”. In some embodiments, the sgRNA can comprise a crRNA covalently linked to a tracrRNA, such as via a linker. In some embodiments, the sgRNA is a Cas9 sgRNA capable of mediating RNA-guided nucleic acid binding and/or cleavage by a Cas9 protein. In some embodiments, the sgRNA is a Cpfl sgRNA capable of mediating RNA-guided nucleic acid binding and/or cleavage by a Cpfl protein. In certain embodiments, the guide RNA comprises a crRNA and tracrRNA sufficient for forming an active complex with a Cas9 protein and mediating RNA-guided nucleic acid binding and/or cleavage. In certain embodiments, the guide RNA comprises a crRNA sufficient for forming an active complex with a Cpfl protein and mediating RNA-guided nucleic acid binding and/or cleavage. In some embodiments, the guide RNA is used to direct RNA cleavage or editing by Cas13. In an embodiment, the RNA-guided DNA endonuclease protein and gRNA targeting leptin can be delivered as the therapeutic agent for treatment of various conditions as further described herein.

CRISPR systems can be used, for example, to decrease or downregulate gene expression of leptin. In an example, the following gRNA sequences uniquely target the LEP gene within the human genome:

(SEQ ID NO: 48)
UCCUCCAAACAGAAAGUCAC
(SEQ ID NO: 49)
CAAACAGAAAGUCACCGGUU
(SEQ ID NO: 50)
CCGGUUUGGACUUCAUUCCU
(SEQ ID NO: 51)
CCCAGGAAUGAAGUCCAAAC
(SEQ ID NO: 52)
CCAAACAGAAAGUCACGGUU
(SEQ ID NO: 53)
CAAACAGAAAGUCACCGGUU
(SEQ ID NO: 54)
CCGGUUUGGACUUCAUUCC
(SEQ ID NO: 55)
UGGAUAAGGUCAGGAUGGGG
(SEQ ID NO: 56)
CAUCUUGGAUAAGGUCAGGA
(SEQ ID NO: 57)
CAUCUUGGAUAAGGUCAGGA
(SEQ ID NO: 58)
GGUCCAUCUUGGAUAAGGUC
(SEQ ID NO: 59)
UGUCUGGUCCAUCUUGGAUA
(SEQ ID NO: 60)
UGCCAGUGUCUGGUCCAUCU
(SEQ ID NO: 61)
AUCCAAGAUGGACCAGACAC.

In an embodiment, the gRNA can target the following sequence (for mouse): gtatccgccaagcagagggtcactggcttgg (SEQ ID NO. 62)

In some embodiments, the method comprises introducing into a subject, cell, or tissue (e.g., adipose tissue) one or more polynucleotides encoding one or more RNA-guided DNA endonucleases. In some embodiments, the method comprises introducing into the subject, cell, or tissue, one or more ribonucleic acids (RNAs) encoding the one or more RNA-guided DNA endonucleases. In some embodiments, the one or more polynucleotides or one or more RNAs is one or more modified polynucleotides or one or more modified RNAs

In some embodiments, the method further comprises introducing into the subject, cell, or tissues, one or more RNA-guided DNA endonucleases, wherein the DNA endonuclease is a protein or polypeptide.

In some embodiments, the method further comprises introducing into the subject, cell, or tissue one or more guide ribonucleic acids (gRNAs). In some embodiments, the one or more gRNAs are single-molecule guide RNA (sgRNAs). In some embodiments, the one or more gRNAs or one or more sgRNAs is one or more modified gRNAs or one or more modified sgRNAs. In some embodiments, the one or more RNA-guided DNA endonucleases are pre-complexed with one or more gRNAs or one or more sgRNAs.

Polynucleotides, such as guide RNA, sgRNA, and mRNA encoding an endonuclease, can be delivered to a cell or a patient by a lipid nanoparticle (LNP).

A LNP refers to any particle having a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses. The endonuclease and sgRNA can be generally combined in a 1:1 molar ratio. Alternatively, the endonuclease, crRNA and tracrRNA can be generally combined in a 1:1:1 molar ratio. However, a wide range of molar ratios may be used to produce an RNP.

A recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes can be from any AAV serotype for which recombinant virus can be derived and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, international patent application publication number WO 01/83692.

In an embodiment, the gRNA can be administered to a patient in an amount of about 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 100, 135, 150, 175, 200, 225, 250, 275, 300, 325, 350, 375, 400, 425, 450, 475, 500, 550, 600, 650, 700, 750, 800, 850, 900, 950, or 1000 mg/kg.

To achieve cell-specific elimination of leptin, a transgenic mouse line with ubiquitously expressed tandem repeats of two small guide RNA (sgRNAs) under the control of the U6 promoter (sgRNA mice) was generated. To achieve adipose tissue specificity, a compound transgenic mouse model that combines APN-rtTA, TRE-Cre and the Rosa26-flox-stop-flox-cas9 alleles was generated. The aforementioned APN-rtTA, TRE-Cre and the Rosa26-flox-stop-flox-cas9 alleles can inducibly activate Cas9 activity in the presence of doxycycline, for example, specifically in mature adipocytes. In combination with the ubiquitously expressed sgRNA transgene, this can allow for the doxycycline-inducible elimination of leptin in the adipose tissues of adult mice (the Cas9-sgLeptin mouse). For the breeding strategy, APN-rtTA, TRE-Cre and rosa26-Cas9, mice were crossed with mice carrying the APN-rtTA and sgLeptin to generate Cas9-sgLeptin mice with expressing all the four transgenes (APN-rtTA, TRE-Cre, Rosa26-Cas9 and sgleptin) and littermate control mice, which express Apn-rtTA, Rosa26-Cas9 and sgleptin without TRE-Cre. All the mice were on a pure C57/BL6 background.

Therapeutic agents of the present disclosure can include one or more agents that can lower or reduce the amount of circulating leptin in a patient or subject. The one or more agents can lower or reduce the amount of circulating leptin by about 30% to about 90% in the patient or subject, or by about 35%, about 40%, about 45%, about 50%, about 55%, about 60%, about 65%, about 70%, about 75%, about 80%, about 85%, or about 90%.

Methods of Treatment

Methods of treating various diseases or conditions are disclosed herein. The methods can comprise administering one or more therapeutic agents, including those disclosed in the present disclosure, to a patient in need thereof. The methods can comprise treating liver fibrosis, treating cancer such as colorectal cancer, inducing or maintaining weight loss, reducing inflammation, reducing tumor growth, reducing or preventing weight gain, and increasing insulin sensitivity, reducing inflammation and fibrosis in patients with COVID-19 infections, inducing breast cancer regression, enhancing effectiveness of PD-1 checkpoint inhibitors, providing metabolic improvements for ciliopathy or Bardet-Biedel Syndrome, providing metabolic improvements for polycystic ovary syndrome (PCOS), among others.

Leptin involves endocrine, paracrine, and autocrine signaling mechanisms and cytokine-mediated inflammatory changes in the body. Therefore, leptin could play a significant role in developing severe COVID-19 infection in patients with obesity. The present disclosure provides methods of reducing inflammation and fibrosis in patients with COVID-19 infections by administering one or more therapeutic agents for lowering the levels of circulating leptin as described herein.

Obesity has been shown to increase breast cancer risk. In addition, leptin can promote the development and progression of breast cancer neoplastic cells by activating/mediating certain pathways, such as the JAK2/STAT3, MAPK, PI3K pathways. The present disclosure provides methods of slowing progression of breast cancer in a patient by administering one or more therapeutic agents as described herein for lowering the levels of circulating leptin.

PD-1 is a checkpoint protein on immune cells called T cells. It can act as a type of “off switch” that helps keep the T cells from attacking other cells in the body. It does this when it attaches to PD-L1, a protein on some normal (and cancer) cells. enhancing effectiveness of PD-1 checkpoint inhibitor. PD-1-mediated T cell dysfunction can be driven, at least in part, by leptin. The present disclosure provides methods of reducing PD-1-mediated T cell dysfunction in a patient by administering one or more therapeutic agents as described herein for lowering the levels of circulating leptin. In some embodiments, one or more therapeutic agents as described herein for lowering the levels of circulating leptin can be combined with the administration of one or more PD-1 checkpoint inhibitors such as pembrolizumab (Keytruda), nivolumab (Opdivo), cemiplimab (Libtayo), JTX-4014 (Jounce Therapeutics), Spartalizumab (PDR001) (Novartis), camrelizumab (SHR1210) (Jiangsu HengRui Medicine Co., Ltd.), sintilimab (161308) (Innovent and Eli Lilly), tislelizumab (BGB-A317), Toripalimab (JS 001), Dostarlimab (TSR-042, WBP-285) (GlaxoSmithKline) INCMGA00012 (MGA012) (Incyte and MacroGenics), AMP-224 (AstraZeneca/MedImmune and GlaxoSmithKline), AMP-514 (MEDI0680) (AstraZeneca), or other PD-1 checkpoint inhibitors. In an embodiment, the therapeutic agent for lowering amounts of circulating leptin and one or more PD-1 checkpoint inhibitors can be administered or simultaneously or sequentially with 1, 10, 20, 30, 40, 50 or 60 minutes between administration, 1, 2, 3, 4, 5, 6, 7, 8, 9, 10, 11 or 12 hours between administration, 0.5, 1, 2, 3, 4, 5, 6, 7 days between administration, or 1, 2, 3 or 4 weeks between administration. Advantageously, the combination of PD-1 checkpoint inhibitors and therapies for lowing circulating leptin as described herein can provide increased efficacy and/or lower the amount of PD-1 checkpoint inhibitor dosage (by about, for example 1, 5, 10% or more).

Ciliopathies are a group of human diseases that involve dysfunction of the cilium. Human patients with mutations in ciliary proteins can exhibit a wide range of phenotypes, one of which is obesity, seen in patients with Bardet-Biedl syndrome (BBS). Obese patients can have a high level of leptin. The present disclosure provides methods of providing metabolic improvements for ciliopathy or Bardet-Biedel Syndrome, including reducing obesity, by administering one or more therapeutic agents for lowering the levels of circulating leptin as described herein.

Polycystic ovarian syndrome (PCOS), a major form of dysovulatory infertility in women, is often associated with obesity and insulin resistance, both of which are features that are linked to leptin and its receptors. Serum level of leptin can be higher in obese women. The present disclosure provides methods of providing metabolic improvements, including reducing obesity, for patients with polycystic ovary syndrome (PCOS) by administering one or more therapeutic agents for lowering the levels of circulating leptin as described herein.

Weight Loss and Maintenance

Methods of inducing weight loss can comprise administering one or more therapeutic agents for lowering the levels of circulating leptin in combination with one or more GLP-1 agonists to a patient or subject in need thereof. The therapeutic agent and one or more GLP-1 agonists can be administered sequentially or simultaneously to the subject or patient. In an embodiment, the therapeutic agent and one or more GLP-1 agonists can be administered sequentially with 1, 10, 20, 30, 40, 50 or 60 minutes between administration, 1, 2, 3, 4, 5, 6, 7, 8, 9, 10, 11 or 12 hours between administration, 0.5, 1, 2, 3, 4, 5, 6, 7 days between administration, or 1, 2, 3 or 4 weeks between administration. The one or more GLP-1 agonists can comprise one or more of liraglutide, exenatide, albiglutide, dulaglutide, semaglutide, or lixisenatide. Many formulations of GLP-1 agonists can be administered, for example subcutaneously or by any other suitable method. Lixisenatide and liraglutide dosing can be, for example, once-daily injections or by any other suitable administration method. Lixisenatide can be administered at, for example, an initial dose of about 1 mcg to about 20 mcg (e.g., about 1, 2, 5, 7, 10, 12, 15, 17, or 20 mcg) subcutaneously (or other suitable delivery method) once, twice, or three times daily for about 7 to about 21 days (e.g., about 7, 14, or 21 days) followed by a maintenance dose increase to about 10-30 mcg (e.g., about 10, 15, 20, 25, 30 mcg) subcutaneously (or other suitable administration method once, twice, or three times daily on the last day of the initial dosing (e.g. day 15) and thereafter. Liraglutide can be administered, for example, at an initial dose of about 0.1 to about 1.0 mg (e.g., 0.1, 0.2, 0.4, 0.6, 0.8, or 1.0 mg) subcutaneously (or other suitable administration method) once, twice, or three times daily for about 7, 14, or 21 days followed by a maintenance dose between about 0.5 and 2.5 mg (e.g. 0.5, 1.0, 1.2, 1.5, 1.8, 2.0, or 2.5 mg) subcutaneously (or other suitable administration method) once, twice, or three times daily. Albiglutide, dulaglutide, and semaglutide dosing can be administered by once daily, once weekly, or once every two week injections (or any other suitable administration). Albiglutide can be administered at an initial dose of about 10-30 mg (e.g., about 10, 15, 20, 25, or 30 mg) subcutaneously once daily, once weekly, or once every other week (or any other suitable administration) followed by a maintenance dose between about 20 and 60 mg (e.g., about 20, 30, 40, 50, or 60 mg) subcutaneously once daily, once weekly, or once every other week (or any other suitable administration. Dulaglutide can be administered at an initial dose of about 0.5 to about 1.5 mg (e.g., about 0.5, 0.75, 1.0, 1.25, 1.5, or 1.75 mg) subcutaneously once daily, once weekly, or once every other week (or any other suitable administration) followed by a maintenance dose between about 0.5 and 2.0 mg (e.g., about 0.5, 0.75, 1.0, 1.25, 1.5, 1.75, and 2.0 mg) subcutaneously once daily, once weekly, or once every other week (or any other suitable administration). Semaglutide can be administered at an initial dose of about 0.1 to about 0.4 mg (e.g., about 0.1, 0.25, 0.3, or 0.4 mg) subcutaneously (or any other suitable administration) once daily, once weekly, or once every other week for about 2, 3, 4, 5, or 6 weeks, then at about 0.1 to about 1.0 mg (e.g., about 0.1, 0.2, 0.5, 0.75, 1.0 mg) subcutaneously (or any other suitable administration) about once a day, once a week, or once every other week, and followed by a maintenance dose between about 0.2 and 2.0 mg (e.g., about 0.2, 0.5, 0.75, and 1.0 mg) subcutaneously (or any other suitable administration) once a day, once weekly, or once every other week. Semaglutide can also come in an oral formulation administered at an initial dose of about 1 to about 5 mg (e.g., about 1, 2, 3, 4, or 5 mg) orally once daily, once weekly or once every other week for about 7, 14, 30, or 45 days (or any other suitable administration); then at about 2-10 mg (e.g., about 2, 3, 4, 5, 6, 7, 8, 9, or 10 mg) orally once daily, once weekly, or once every other day; the maintenance dose can be between about 5 and 20 (e.g. about 5, 7, 10, 12, 15, 17, and 20 mg/day). Exenatide dosing can be, for example, once daily, twice daily or once-weekly injections. Exenatide twice-daily (immediate-release) can be administered at an initial dose of about 2 to about 7 mcg (e.g., about 2, 3, 4, 5, 6, or 7) mcg subcutaneously twice daily within a 60-minute period before morning and evening meals and can be followed by a maintenance dose between 1 and 10 mcg (e.g., about 1, 2, 3, 4, 5, 6, 7, 8, 9, or 10 mcg) subcutaneously twice daily (or any other suitable administration). Exenatide once-weekly (extended release) can be administered at an initial dose of about 1, 2, 3, or mg subcutaneously once-weekly. Methods of inducing weight loss can comprise administering one or more therapeutic agents with one or more GLP-1 agonists and can further comprise removing the one or more GLP-1 agonists from the treatment regimen after a desired or target weight level is achieved by the patient or subject, while the administration of the therapeutic agent is continued. The desired or target weight level can be any weight level desired or targeted by the subject or patient, or can be a target weight level that achieves a healthy BMI (i.e., 18.5-24.9). A target weight level can be a loss of about 5, 10, 20, 30, 40% or more of a starting weight level. A target weight level can be a loss of about 5, 10, 20, 30, 40, 50, 60, 70, 80, 90, 100 pounds or more as compared to a starting weight. The one or more GLP-1 agonists can be removed from the treatment regimen after the target weight level is achieved. In an embodiment, a method of reducing the amount of a GLP-1 agonist delivered to a patient is provided by sequentially or simultaneously delivering the GLP-1 agonist with a therapeutic agent that can reduce the amount of circulating leptin in the patient. The dose of GPL-1 agonist can be reduced by about 1 to 50% (e.g., a reduction of about 1, 5, 10, 15, 20, 30, 40, 50% or more) of the standard dose. A patient can experience weight gain associated with GPL-1 agonist (such as liraglutide) withdrawal. In an embodiment, a method of reducing or slowing weight gain associated with GPL-1 agonist (e.g., liraglutide) withdrawal is provided by sequentially or simultaneously delivering the GLP-1 agonist with a therapeutic agent that can reduce the amount of circulating leptin in the patient.

The lack of leptin changes during fasting, when basal insulin and glucose levels were maintained at basal levels in a patient, can suggest that insulin and/or glucose may play a role in the regulation of leptin release. Metabolism can move from using glucose to burning fat when there is a drop in both insulin and leptin level. Leptin can also independently lower blood glucose levels, particularly in hyperglycemic models of leptin or insulin deficiency. In an embodiment, a method of reducing fasting glycemia (which occurs when blood glucose levels in the body are elevated during periods of fasting) and/or improving glucose tolerance (improving the ability to dispose a glucose load) is provided by sequentially or simultaneously delivering the GLP-1 agonist with a therapeutic agent that can reduce the amount of circulating leptin in the patient. In an embodiment, the therapeutic agent and one or more GLP-1 agonists can be administered sequentially with 1, 10, 20, 30, 40, 50 or 60 minutes between administration, 1, 2, 3, 4, 5, 6, 7, 8, 9, 10, 11 or 12 hours between administration, 0.5, 1, 2, 3, 4, 5, 6, 7 days between administration, or 1, 2, 3 or 4 weeks between administration.

Other embodiments provide methods of maintaining weight loss. Methods of maintaining weight loss can comprise administering one or more therapeutic agents that can reduce or lower circulating leptin to a patient or subject after weight loss. That is, after a subject attains a specified amount of weight loss, one or more therapeutic agents described herein can be administered to maintain that weight loss. A weight loss can be a loss of about 5, 10, 20, 30, 40% or more of a starting weight resulting in a target weight. A weight loss can be a loss of about 5, 10, 20, 30, 40, 50, 60, 70, 80, 90, 100 pounds or more as compared to a starting weight resulting in a target weight. In an embodiment, a subject maintains the weight loss for 1, 2, 3, 4, 5, 6, or more months or 1, 2, 3 or more years. A maintained weight loss means that the subject does not gain more than about 1, 2, 3, 4, 5, 10, or 20% of their target weight.

In some embodiments, a therapy to reduce circulating leptin as described herein is administered to a patient who has recently (for example, in the last 1, 2, 3, 4, 6, 8, 10 or 12 months) lost 5% or more (e.g., about 5, 10, 20% or more) of their bodyweight. The therapy can comprise administering one or more therapeutic agents that can reduce or lower circulating leptin to the patient who has recently lost weight for 1 or 2 weeks, or 1, 2, 4, 6, 8, 10, 12 months or more. The patient does not gain more than about 3%, 5%, 10% of their body weight over a 1, 2, 4, 6, 8, 10, or 12 month time frame or more after or during administration of the leptin therapy.

Methods of the present disclosure can comprise methods of reducing or preventing weight gain, for example, weight gain resulting directly or indirectly from administration of an anti-psychotic drug or medication. Such drugs can be olanzapine (2.5 mg, 5 mg, 7.5 mg, 10 mg, 15 mg, 20 mg, 210 mg/vial, 300 mg/vial, or 400 mg/vial), zotepine (25 mg three times a day, or a gradual incremental increase not exceeding 100 mg three times a day) or clozapine (25 mg, 50 mg, 100 mg, 200 mg, or 50 mg/mL oral suspension). Methods of reducing or preventing weight gain can comprise administering one or more anti-psychotic drugs or medications in combination with one or more therapeutic agents that can reduce or lower circulating leptin to a patient or subject, wherein the one or more therapeutic agents and one or more anti-psychotic drugs can be administered sequentially or simultaneously to the patient or subject.

In an embodiment, the therapeutic agent and one or more anti-psychotic drugs can be administered sequentially with 1, 10, 20, 30, 40, 50 or 60 minutes between administration, 1, 2, 3, 4, 5, 6, 7, 8, 9, 10, 11 or 12 hours between administration, 0.5, 1, 2, 3, 4, 5, 6, 7 days between administration, or 1, 2, 3 or 4 weeks between administration.

In some embodiments, a therapy is administered to a patient who has gained weight directly or indirectly resulting from administration of an anti-psychotic drug or medication, wherein the therapy comprises sequentially or simultaneously administering one or more anti-psychotic drugs and one or more therapeutic agents that can reduce or lower circulating leptin to the patient. The patient does not gain more than about 3%, 5%, 10% of their body weight over a 1, 2, 4, 6, 8, 10, or 12 month time frame or more after or during administration of the leptin therapy. In some embodiments a patient is administered an anti-psychotic drug and a therapeutic agent that can reduce the amount of circulating leptin, wherein the subject does not gain more than 3% (include a range) of their body weight over X time frame.

Cardiovascular Disease

The present disclosure provides a treatment of obesity-associated cardiovascular disorders by lowering the levels of leptin, for example visceral fat-derived leptin. In an embodiment, a therapy is administered to a patient who has obesity-associated cardiovascular disorders, wherein the therapy comprises administering one or more therapeutic agents that can reduce or lower circulating leptin to the patient. In one embodiment, the leptin is visceral fat-derived leptin. In an embodiment, the present disclosure provides a treatment of one or more symptoms of obesity-associated cardiovascular disorders comprising administering one or more therapeutic agents that can reduce or lower circulating leptin to the patient as described herein, wherein the one or more symptoms comprise chest tightness or pressure, difficulty catching one's breath, dizziness or fainting, fatigue, fluid build up, heart palpitations (heart pounding or racing, pain or numbness in one's legs or arms, and/or abdominal pain, nausea, and/or vomiting).

Methods of Cancer Treatment

Methods of treating cancer can comprise administering one or more therapeutic agents that can reduce or lower circulating leptin to a patient or subject in need thereof. The cancer can comprise one or more of colorectal cancer, cancer of the breast, prostate, head, neck, eye, mouth, throat, esophagus, bronchus, larynx, pharynx, chest, bone, lung, colon, rectum, stomach, liver (for example, hepatocellular carcinoma), bladder, uterus, cervix, ovaries, vagina, testicles, skin, thyroid, blood, lymph nodes, kidney, liver, intestines, pancreas, brain, central nervous system, adrenal gland, skin or a leukemia (such as acute lymphoblastic leukemia), lymphoma, or any other cancer. In some embodiments, a cancer patient is administered a therapeutic agent that can reduce circulating leptin concentrations as described herein such that one or more symptoms of cancer are reduced or eliminated. In an embodiment, the therapeutic agent can kill cancerous cells, reduce tumor size, or reduce tumor growth. In some embodiments, the cancer can be, for example, liver cancer, colorectal cancer or leukemia such as acute lymphoblastic leukemia.

Insulin Sensitivity

Insulin sensitivity refers to how sensitive the body's cells are in response to insulin. An embodiment provides methods of increasing insulin sensitivity. Glycated hemoglobin, which is a form of hemoglobin that is chemically linked to sugar, can be measured to reflect average blood glucose levels over a time period, for example, an eight (8) to twelve (12) week period. A decrease in blood sugar levels can be reflected when hemoglobin A1c (HbA1c) is lowered, for example, by at least 1% point (e.g., at least 1, 2, 3, 4, 5, 6, 7, 8, 9, 10, 15, 20 or more % points). In an embodiment, insulin sensitivity can be increased quickly in a patient, for example, within about 24, 12, 8, 4 or fewer hours. Insulin sensitivity can be measured using, for example, a hyperinsulinemic euglycemic clamp (HEIC). A HEIC technique works by perfusing or infusing insulin as a way to quantify how sensitive the tissue is to insulin. In an HEIC technique plasma insulin concentration is acutely raised and maintained by a continuous infusion of insulin. At the same time, the plasma glucose concentration is held constant at basal levels by a variable glucose infusion. When a steady state is achieved, the glucose infusion rate is equal to the glucose uptake by all the tissues in the body, serving as a measure of tissue insulin sensitivity thus quantifying insulin resistance. Other insulin resistance tests include variations on a glucose tolerance test (GTT), which determine how quickly glucose is cleared from the blood. One GTT is the oral glucose tolerance test (OGTT) used to determine how quickly glucose is cleared from the body and therefore quantify insulin resistance. In the OGTT, a standard dose of glucose is ingested orally, and blood glucose levels are measured at fixed time intervals afterwards. Methods of increasing insulin sensitivity can comprise administering one or more therapeutic agents that can reduce or lower circulating leptin as described herein to a patient or subject in need thereof.

Liver Disease: Liver Fibrosis, Nonalcoholic Fatty Liver Disease (NAFLD) and Nonalcoholic Steatohepatitis (NASH)

Excess fat accumulation in the liver is a health threat globally. Sustained liver injury leads to progressive fibrosis (formation of permanent scar tissue) and cirrhosis (irreversible scaring of liver tissue). Nonalcoholic fatty liver disease (NAFLD), also known as metabolic (dysfunction) associated fatty liver disease (MAFLD), is characterized by excessive fat build-up in the liver cells (hepatocytes) that occurs without alcohol use. Typical liver tissue abnormalities include fatty deposits, tissue degeneration, varying degrees of inflammation, cell degeneration, fibrosis, cirrhosis, elevation of free fatty acids, and other such abnormalities. NAFLD can refer to a spectrum of liver diseases including steatosis, nonalcoholic fatty liver (NAFL), and nonalcoholic steatohepatitis (NASH). NASH is further characterized by liver inflammation and is therefore considered more dangerous than NAFL. NASH and NAFL occur in both men and women, but it appears in women more often. NASH and NAFL are prevalent among obese individuals.

Leptin is associated with metabolic disorders, which can predispose one to NASH and NAFL. Methods of treating NASH and NAFL can comprise administering one or more therapeutic agents as described herein, which can reduce or lower circulating leptin in a patient or subject in need thereof, such that one or more symptoms of NASH or NAFL are reduced or eliminated.

Leptin is thought to increase hepatic steatosis, inflammation and fibrosis. Leptin can have a pro-inflammatory role that contributes to the development of fibrosis. Circulating leptin level can be increased in liver cirrhosis. Methods of treating liver fibrosis, liver cirrhosis, or liver cancer are provided herein, comprising administering one or more therapeutic agents as described herein, which can reduce or lower circulating leptin in a patient or subject in need thereof, such that one or more symptoms of liver fibrosis, liver cirrhosis or liver cancer are reduced or eliminated. Such symptoms can comprise inflammation.

Pharmaceutical Compositions

Pharmaceutical compositions useful herein contain therapeutic agents as disclosed herein in a pharmaceutically acceptable carrier, optionally with other pharmaceutically inert or inactive ingredients. Therapeutic agents of the present disclosure can be present in a single composition or can be combined with one or more excipients and/or other therapeutic agents.

The pharmaceutical compositions can comprise an amount of a therapeutic agent that is effective for reducing or lowering circulating leptin levels in a patient or subject. The dosage of the therapeutic agent to achieve a therapeutic effect will depend on the formulation, age, weight and sex of the patient and route of delivery. It is also contemplated that the treatment and dosage of the agent can be administered in unit dosage form and that one skilled in the art would adjust the unit dosage form accordingly to reflect the relative level of activity. The decision as to the particular dosage to be employed (and the number of times to be administered per day) is within the discretion of the ordinarily-skilled physician, and can be varied by titration of the dosage to the particular circumstances to produce the desired therapeutic effect. The therapeutically effective amount of the agent can be determined by the attending physician and depends on the condition treated, the agent administered, the route of delivery, the age, weight, severity of the patient's symptoms and response pattern of the patient.

The therapeutically effective amounts can be provided on regular schedule, i.e., daily, weekly, monthly, or yearly basis or on an irregular schedule with varying administration days, weeks, months, etc. Alternatively, the therapeutically effective amount to be administered can vary. The therapeutically effective amount for the first dose can be higher than the therapeutically effective amount for one or more of the subsequent doses. The therapeutically effective amount for the first dose can be lower than the therapeutically effective amount for one or more of the subsequent doses. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The therapeutically effective amounts described herein refer to total amounts administered for a given time period; that is, if more than one agent or a pharmaceutically acceptable salt thereof is administered, the therapeutically effective amounts correspond to the total amount administered.

The pharmaceutical compositions containing therapeutic agents of the present disclosure can be formulated neat or with one or more pharmaceutical carriers for administration. The amount of the pharmaceutical carrier(s) is determined by the solubility and chemical nature of the agent, chosen route of administration and standard pharmacological practice. The pharmaceutical carrier(s) can be solid or liquid and can incorporate both solid and liquid carriers. A variety of suitable liquid carriers is known and can be readily selected by one of skill in the art. Such carriers can include, e.g., an aqueous PBS buffer. Similarly, a variety of solid carriers and excipients are known to those of skill in the art. The agents of the present disclosure can be administered by any route, taking into consideration the specific condition for which it has been selected. The agent(s) can be delivered by injection, ocularly, transdermally, intravascularly, subcutaneously, intramuscularly, sublingually, intracranially, epidurally, rectally, and vaginally, among others.

Although the agents of the present disclosure can be administered alone, they can also be administered in the presence of one or more pharmaceutical carriers that are physiologically compatible. The carriers can be in dry or liquid form and are pharmaceutically acceptable. Liquid pharmaceutical compositions are typically sterile solutions or suspensions. When liquid carriers are utilized for parenteral administration, they are desirably sterile liquids. Liquid carriers are typically utilized in preparing solutions, suspensions, emulsions, syrups and elixirs. In one embodiment, the agent disclosed herein is dissolved a liquid carrier. In another embodiment, the agent is suspended in a liquid carrier. One of skill in the art of formulations would be able to select a suitable liquid carrier, depending on the route of administration. The agent can alternatively be formulated in a solid carrier.

The composition can also be sub-divided to contain appropriate quantities of the agent. For example, the unit dosage can be packaged compositions, e.g., packeted powders, vials, ampoules, prefilled syringes or sachets containing liquids.

Examples of excipients which can be combined with one or more therapeutic agents include, without limitation, adjuvants, antioxidants, binders, buffers, coatings, coloring agents, compression aids, diluents, disintegrants, emulsifiers, emollients, encapsulating materials, fillers, flavoring agents, glidants, granulating agents, lubricants, metal chelators, osmo-regulators, pH adjustors, preservatives, solubilizers, sorbents, stabilizers, sweeteners, surfactants, suspending agents, syrups, thickening agents, or viscosity regulators.

In another embodiment, the compositions can be administered by a sustained delivery device. “Sustained delivery” as used herein refers to delivery of an agent which is delayed or otherwise controlled. Those of skill in the art know suitable sustained delivery devices. For use in such sustained delivery devices, the therapeutic agent is formulated as described herein.

Also provided herein are kits or packages of pharmaceutical formulations containing the agents and/or compositions described herein. The kits can be organized to indicate a single formulation or combination of formulations to be taken at a desired time. The kit contains packaging or a container with the therapeutic agent(s) formulated for the desired delivery route. Suitably, the kit contains instructions on dosing and an insert regarding the active agent(s). Optionally, the kit can further contain instructions for monitoring circulating levels of product and materials for performing such assays including, e.g., reagents, well plates, containers, markers or labels, and the like. Such kits are readily packaged in a manner suitable for treatment of a desired indication. For example, the kit can also contain instructions for use of a spray pump or other delivery device. Other suitable components to include in such kits will be readily apparent to one of skill in the art, taking into consideration the desired indication and the delivery route.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

Any single term, single element, single phrase, group of terms, group of phrases, or group of elements described herein can be each be specifically excluded from the claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### Example 1

Surgery was performed on single housed diet-induced obese mice with a body weight of approximately or about 40 g. After one week of recovery, the body weight of the mice were similar to the body weight before surgery. A first hyperinsulinemic euglycemic (HIEC) clamp was performed on all the mice without any treatment. Based on this initial screening, the mice were grouped into two different groups with similar glucose infusion rates (GIR). One day after the first clamp, the mice were injected either with a control antibody (CtrlAB) or a leptin neutralizing antibody (LepAB). The next day, all the mice were food restricted for 4 hrs in the morning. Then the second HIEC clamp was performed on the same mice. Glycemia and GIR were recorded during the clamp processes.

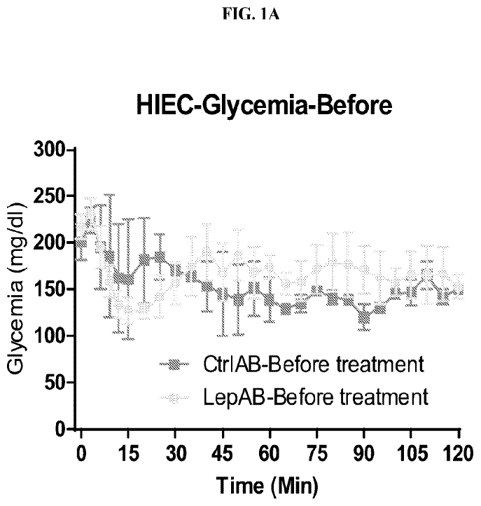

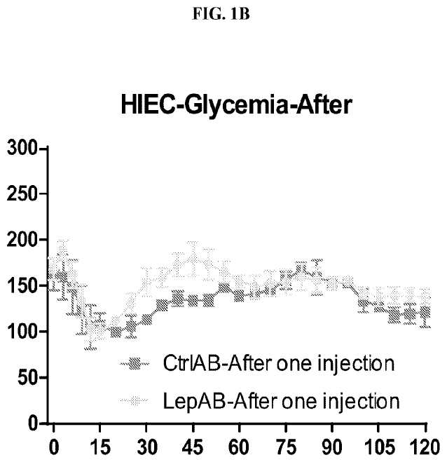

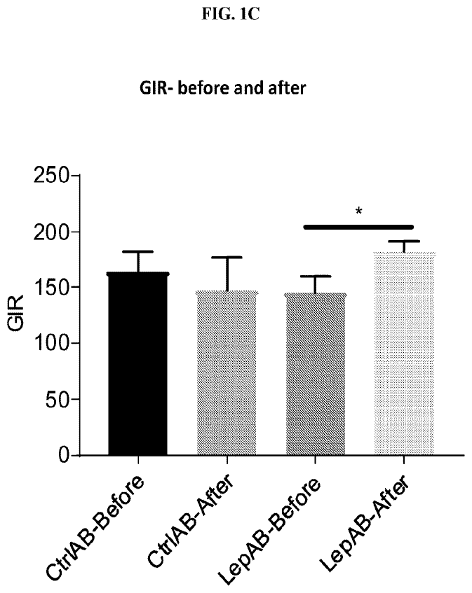

CtrlAB had little or no effect in GIR, and, surprisingly and unexpectedly, after acute LepAb treatment, GIR was greatly increased, which indicates increased insulin sensitivity. A single injection of LepAB increases insulin sensitivity in obese mice. See FIGS. 1A, 1B and 1C.

### Example 2

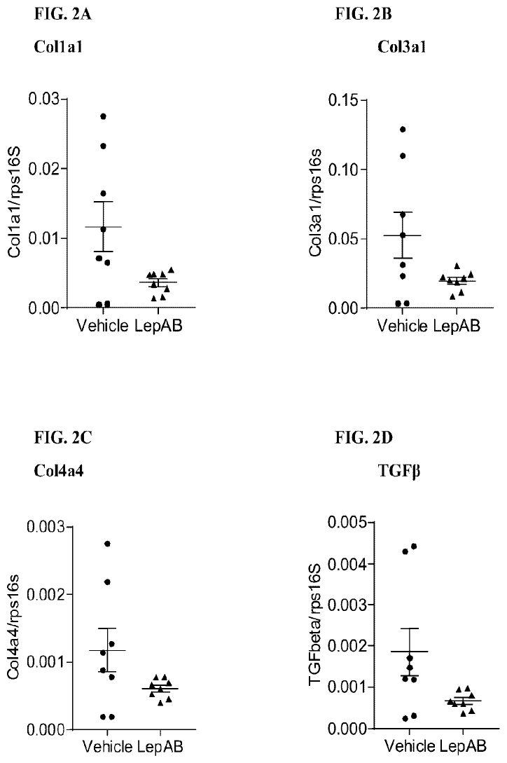

Liver fibrosis is a liver disease that can further develop into liver cirrhosis and liver cancer. Example 2 shows unexpected and surprising effect of the LepAB in liver fibrosis, for example, that LepAB can alleviate liver fibrosis in Mup-uPA mouse model (FIG. 2).

A new mouse model, (Mup-uPA), that develops liver fibrosis and liver cancer upon challenge with high fat diet for 20 weeks was used. Mup-uPA mice were placed on high-fat diet (HFD) for 18 weeks. Subsequently, the mice were allocated into two groups: one group received control antibody, and the other group received lepAB at a dose of about 5 mg/kg body weight, and the treatment was sustained for 2 weeks. Then the mice were euthanized for liver analysis. Analysis of euthanized Mup-uPA mice indicated that expression of three fibronic genes, Col1a1, Col3a1, and Col4a4, and one cytokine, TGF-β, was greatly reduced in LepAB treated mice as compared to vehicle treated mice (FIGS. 2A-D).

LepAB treatment can reduce expression of fibronic genes, indicating protective effects in reversing liver fibrosis. Therefore, the methods can be used to treat, for example, nonalcoholic fatty liver disease (NAFLD) and nonalcoholic steatohepatitis (NASH), among others.

### Example 3

GLP-1 agonists (such as liraglutide) can show effects in inducing weight loss and improving glucose tolerance, and they are FDA approved as weight loss drugs and type-2 diabetes. Example 3 surprisingly and unexpectedly indicates the effectiveness of lepAB in inducing weight loss and improving glucose tolerance, as well as a synergistic effect of the combination of GLP-1 agonist and LepAB for inducing weight loss and/or anti-diabetes effects. The combination of GLP-1 agonists and LepAB can allow for a reduction of GLP-1 agonist doses that are commonly associated with various side effects.

24 wild-type (WT) mice were placed on a 60% high-fat diet (HFD) for 20 weeks to reach a body weight of about 55 g. According to similar body weight, food intake and glucose tolerance, the mice were categorized into 4 groups: Group 1 received PBS and control AB; Group 2 received PBS and LepAB; Group 3 received liraglutide and Ctrl AB; Group 4 received liraglutide and LepAB. The liraglutide was administered on a daily basis, while LepAB and CtrlAB were given every other day. The treatment lasted for 2 weeks. Then, an oral glucose tolerance test (OGTT) was performed and then all the mice were euthanized for blood and tissues, used for further analysis.

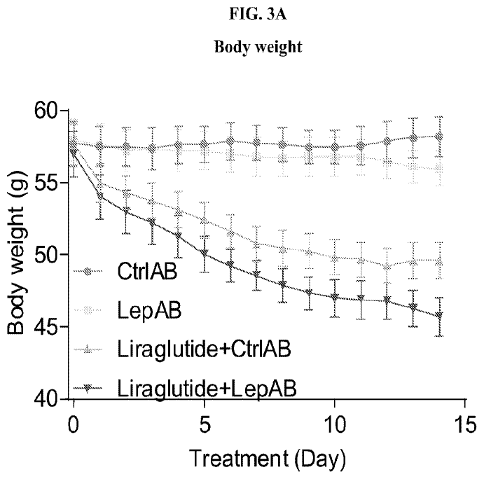

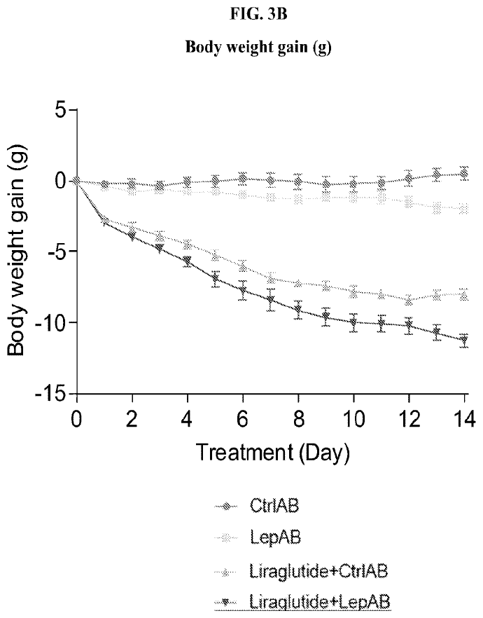

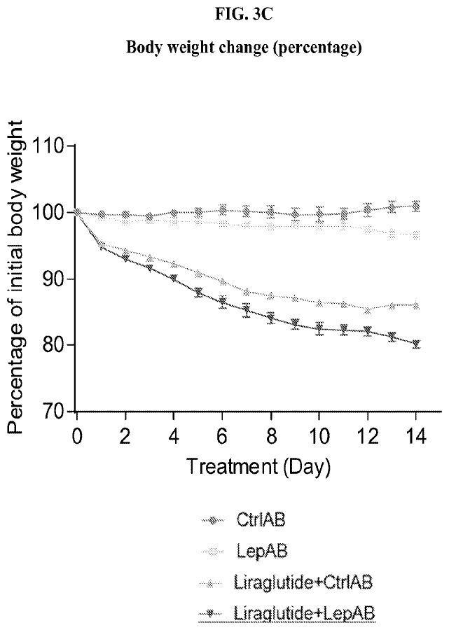

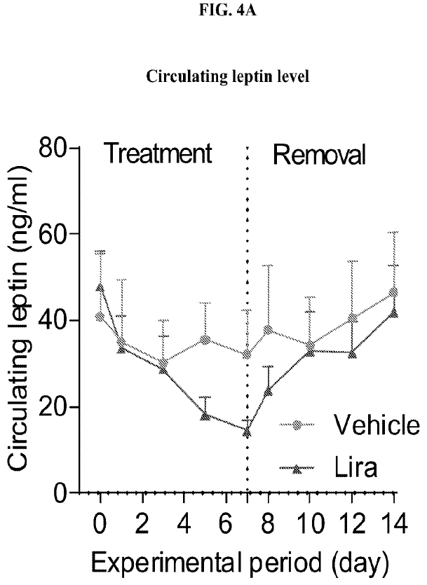

GLP-1 and LepAB demonstrated unexpected and surprising synergistic effects in inducing weight loss. There were no significant changes in body weight for Group 1 mice, while Group 2 mice lost some weight over the 15 day treatment period (FIG. 3A). Group 3 and Group 4 mice both showed significantly more weight loss than Group 1 and Group 2 mice over the same 15 day period, demonstrating a synergistic effect of Liraglutide, a GLP-1 agonist, and LepAB (FIG. 3A). Group 1 mice gained weight over the 15 day period, while Group 2, Group, 3, and Group 4 mice had a negative body weight gain over the same 15 day period (FIG. 3B). The Group 3 and Group 4 mice lost significantly more weight than the Group 2 mice over the same 15 day period, demonstrating a synergistic effect of Liraglutide, a GLP-1 agonist, and LepAB (FIG. 3B). Body weight change as a percentage showed a similar pattern: Group 1 mice ultimately gained weight, Group 2 mice lost some weight, and Group 3 and 4 mice lost significantly more weight than Group 1 and 2 mice (FIG. 3C). See FIGS. 3A, 3B and 3C.

### Example 4

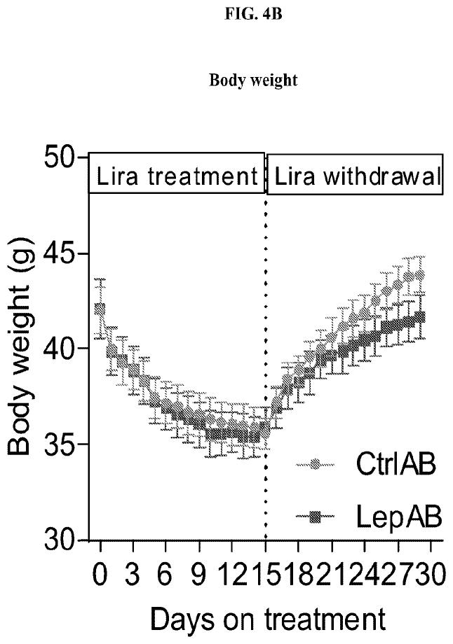

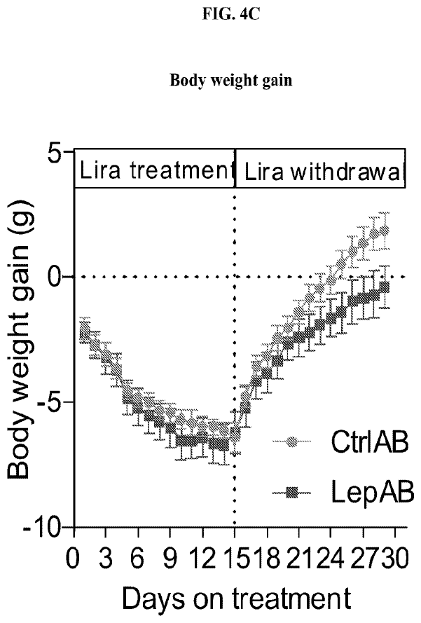

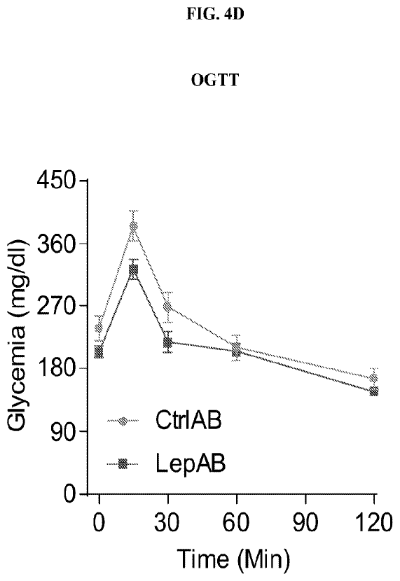

Example 4 demonstrates surprising and unexpected effects of LepAB in maintaining of GLP-1 induced weight loss. Upon withdrawal of liraglutide from a treatment group, the patients underwent a rapid weight rebound and reached a body weight even higher than the starting weight. Immediately after withdrawal, an increase in circulating leptin was observed, and this increased leptin level can contribute to the rebound of body weight. Reducing circulating leptin levels by LepAB administration can help in maintaining the weight loss, induced by liraglutide, even after washout of liraglutide. First, it was determined that circulating leptin levels rebound immediately after removing liraglutide. Circulating leptin levels were measured for 14 days in diet-induced obese mice treated with vehicle or liraglutide for the first 7 days (FIG. 4A). Mice treated with liraglutide experienced an initial decrease in circulating leptin levels compared to the onset of treatment and to the vehicle group (FIG. 4A). Following treatment removal at day 7, circulating leptin levels rebounded reaching the level of the control group and onset of treatment by day 14 (FIG. 4A). This initial experiment demonstrated that alone, liraglutide leads to rapid weight rebound.

In Example 4, 16 diet-induced obese mice were treated with liraglutide at a dose of about 0.1 mg/kg body weight to reach maximum weight loss. Liraglutide was removed from the treatment after 15 days, at which point half of the mice received control antibody. The other half of the mice received LepAB for another two weeks. Body weight and food intake were measured during each injection. Glucose tolerance was performed at the end of the experiment.

In liraglutide withdrawal, circulating leptin level is increased, which can be associated with weight gain (FIG. 4A). Reducing circulating leptin level by LepAB administration slowed weight gain (FIGS. 4B-C) and reduced fasting glycemia and improved glucose tolerance. See FIGS. 4A, 4B, 4C and 4D.

### Example 5

Olanzapine is a novel antipsychotic agent with broad efficacy. However, the side effects of olanzapine can include weight gain. A rapid increase in circulating leptin levels can occur prior to weight gain, and increased leptin can be the driver for weight gain. Reducing circulating leptin by LepAB can help reduce body weight gain during exposure to anti-psychotics. Example 6 shows surprising and unexpected effects of LepAB in counteracting anti-psychotic-induced weight gain.

While both men and women can gain weight as a side effect of antipsychotic (AP) treatment, studies in mice have found that female mice are susceptible to weight gain. At the start of the study, female mice were randomized (n=6/group) to receive either the 45% HFD with or without olanzapine (54 mg/kg, D161110301, Research Diets) and LepAB. This model of administering olanzapine in 45% HFD to initiate weight gain has been used in other studies. During the treatment period with olanzapine and LepAB, body weight and food intake were measured.

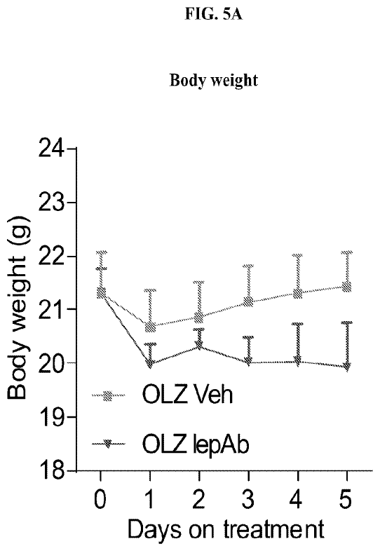

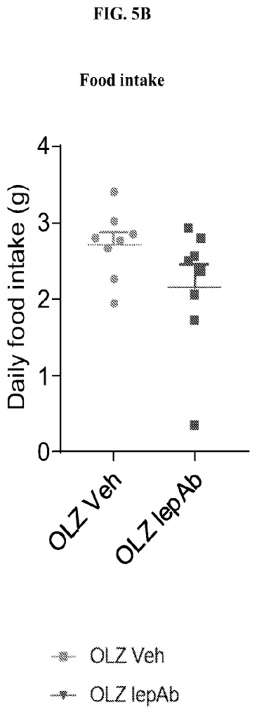

LepAB treatment can prevent olanzapine-induced weight gain, and this effect can mediate through reduced food intake. Body weight gain over time was less for olanzapine and LepAB treated female mice than for just olanzapine treated mice (FIG. 5A). Furthermore, daily food intake was less for olanzapine and LepAB treated female mice than for just olanzapine treated mice (FIG. 5B)

### Example 6

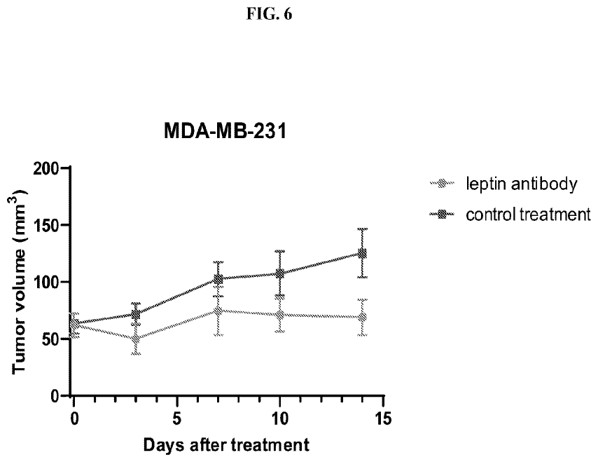

Example 6 shows the surprising and unexpected effects of LepAB in preventing and/or reversing breast cancer. Human MDA-MB-231 breast cancer cells were implanted at a dose of about 2 million cells per mouse into nude mice (n=10 total). After implantation, the mice were allowed to recover for about 3 weeks, allowing the tumor size to reach more than 50 mm³. According to similar tumor size, the mice were allocated into two groups, treated either with control antibody or LepAB at a dose of about 100 ug/mouse. The injection was done twice per week (Monday and Thursday). During each injection, tumor size was measured. Leptin antibody treated mice showed a significant reduction in tumor volume as compared to control treatment mice, demonstrating the effects of LepAB in preventing and/or reversing breast cancer (FIG. 6).

Circulating leptin can be a driver for tumor growth. Reducing circulating leptin level by LepAB antibody completely abolished tumor growth.

### Example 7

Example 7 shows the surprising and unexpected effect of LepAB in acute leukemia. Fasting selectively blocks the development of acute lymphoblastic leukemia, and leptin signaling can be involved in this phenomenon. Also, fasting can induce a rapid fall in circulating leptin levels. This example shows that reduced circulating leptin levels can trigger increased leptin receptor expression to exert its effect on acute leukemia. Reducing circulating leptin levels with LepAB can recapture the effects of fasting on blocking development of acute lymphoblastic leukemia.

In Example 7, 6-8-week-old male mice were used and randomly allocated to each group. Lin⁻ cells were isolated from the fetal liver or bone marrow of wild-type mice, and infected with an oncogene-IRES-GFP(YFP) expressing retrovirus. Infected mouse Lin⁻ cells (300,000) were transplanted into lethally irradiated (900 cGy) C57BL/6 mice. Fluorescence-activated cell sorting (FACS) was used to isolate GFP⁺ or YFP⁺ BM cells from primary recipient mice and 3,000 cells (AML) or 10,000 cells (B- or T-ALL) together with 3×10⁵ normal BM cells were transplanted into lethally irradiated recipients.

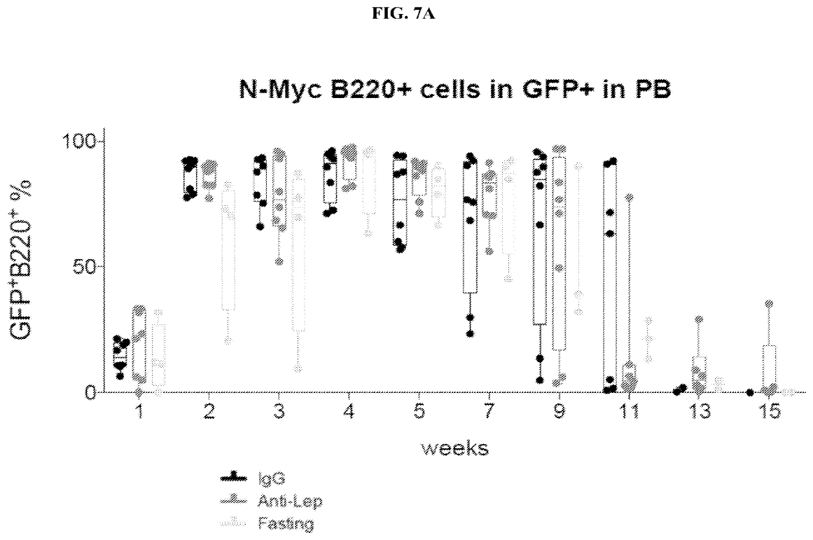

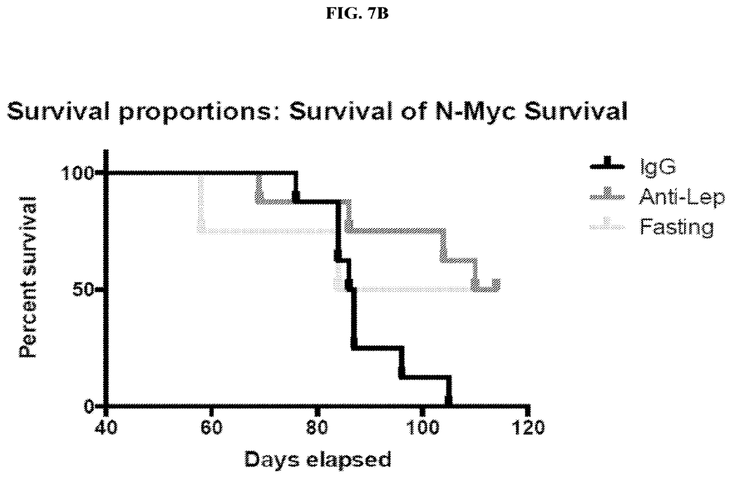

Fasting and LepAB treatment groups increased the percent survival of N-Myc proto-oncogene protein over time as compared to the control group, IgG (FIG. 7B). FIGS. 7A and 7B show that, similar to fasting, LepAB can block development of acute leukemia and can increase life span.

### Example 8

Example 8 demonstrates neutralizing leptin antibodies and their use in cardiovascular disease. As a pleiotropic hormone, leptin can be one of the adipokines to mediate obesity-associated cardiovascular disorders. The positive association between hyperleptinemia and unfavorable outcomes in cardiovascular disorders can suggest a role of leptin in the progression of cardiovascular disorders. The present disclosure demonstrates that hyperleptinemia can be a driving force for diet-induced obesity, and partial leptin reduction can elicit significant weight loss in diet-induced obese mice. Based on these observations, a new concept is proposed that “less leptin is more” under obesogenic conditions and also cardiovascular function. As leptin's action on cardiovascular function is mediated via both central and peripheral mechanisms, and partial leptin reduction strategy can restore leptin action centrally and peripherally, the strategy of leptin reduction has implications in treating cardiovascular disorders. The effects of hyperleptinemia in cardiovascular function is dependent on leptin responsive states (leptin resistance vs leptin sensitivity). A partial leptin reduction presents a novel therapeutic approach for obesity-associated cardiovascular disorders.

Visceral fat has a unique anatomical location and close-knit interactions with the heart and vasculature. Visceral adiposity is independently associated with an elevated risk of cardiovascular disorders. As obesity develops, leptin derived from visceral fat becomes a major source for circulating leptin. Thus, visceral fat-secreted leptin reaches high levels locally to directly activate functional leptin receptors (long-form) in the heart and vasculature to promote local inflammation and disease progression. Mice with specific deletion and overexpression of leptin in visceral fat have provided a better understanding of leptin's physiological role on cardiovascular function. In addition, deletion of leptin exclusively in visceral fat can create an independent mouse model of partial leptin reduction, allowing the beneficial effects of partial leptin reduction in cardiovascular disorders.

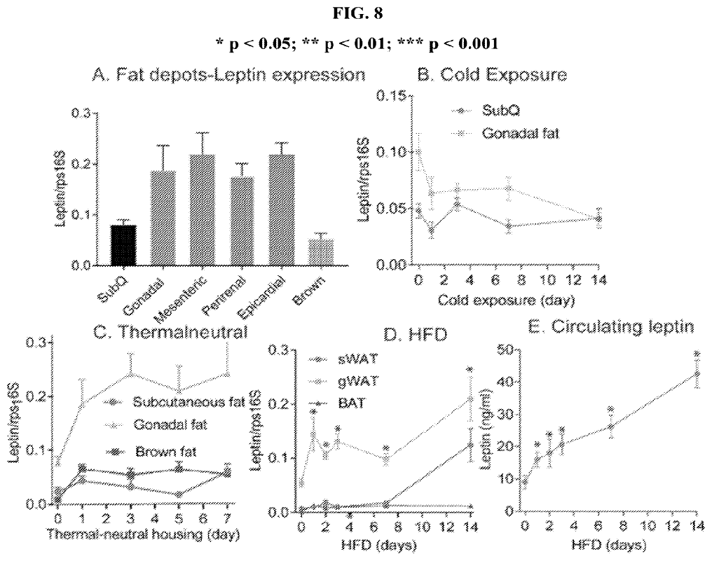

Example 8 and FIGS. 8A to 8E focus on visceral fat-derived leptin and provide the following observations: 1) the positive association between visceral adiposity and cardiovascular disease can help in identifying the causative factors in mediating the adverse metabolic consequence of visceral fat; 2) due to its unique anatomical location and close-knit interactions with inner organs, visceral fat-secreted adipokines or cytokines are more likely to exert their autocrine/paracrine effect to promote various metabolic disorders; 3) visceral fat-derived leptin (which looks the same as subcutaneous fat-derived leptin, but can be differentiated by knocking it out selectively in visceral fat tissue) can be one of the strongest candidates, as visceral fat-derived leptin not only functions as an adipokine to exert its “endocrine” effects in regulating energy homeostasis, but it also acts as a proinflammatory cytokine to fulfill its autocrine/paracrine roles in closely juxtaposed inner organs, including the heart and vasculature; 4) a unique leptin signature is observed in visceral fat depots, distinct from subcutaneous and brown fat (FIG. 8A). The intrinsic expression of lep gene in visceral fat depots (gonadal fat, mesenteric fat, perirenal fat and epicardial fat) is much higher than in subcutaneous fat and brown fat (FIG. 8A). In response to acute physiological stimuli, such as acute cold exposure (FIG. 8B), thermoneutral housing (FIG. 8C) and short-term high fat diet (HFD) (FIGS. 8D and 8E), acute changes in lep gene expression occur only in visceral fat, but not in subcutaneous and brown fat. Thus, Example 8 and FIGS. 8A to 8E demonstrate that the altered circulating leptin level in response to acute high fat feeding is mainly derived from visceral fat. Example 8 demonstrates that obesity-associated cardiovascular disorders can be treated by lowering the levels of circulating leptin, for example visceral fat-derived leptin.

### Example 9

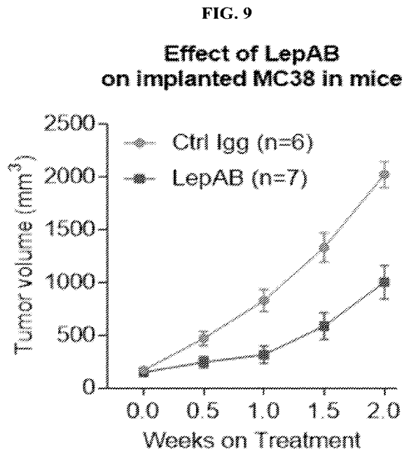

Example 9 and FIG. 9 demonstrate neutralizing leptin antibodies and effects on colorectal cancer, in particular the effects of LepAB on MC38-associated tumor growth. MC38 cells are a colorectal tumor cell line. One (1) million MC38 cells were implanted into C57/BL6 wt mice. One week after implantation, tumor volumes were recorded and split into two equal groups and treated with either control antibodies or anti-leptin antibodies. Tumor volume was measured during antibody treatment. FIG. 9 shows the tumor volume (mm³) after treatment with either control antibodies or anti-leptin antibodies over time.

1. A method of treating liver disease, liver fibrosis, liver cirrhosis, maintaining weight loss, treating cancer, treating colorectal cancer, treating acute lymphoblastic leukemia, treating cardiovascular disease or one or more symptoms of cardiovascular disease, reducing fasting glycemia, improving glucose tolerance, reducing an amount of GLP-1 agonist delivered to a subject, increasing insulin sensitivity within 24 or fewer hours, reducing inflammation and fibrosis in COVID-19 infections, inducing breast cancer regression, enhancing effectiveness of PD-1 checkpoint inhibitors, providing metabolic improvements for ciliopathy or Bardet-Biedel Syndrome, providing metabolic improvements for polycystic ovary syndrome (PCOS), and combinations thereof, comprising administering a therapeutic agent for lowering circulating leptin to a subject in need thereof,
wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

2. The method of claim 1, wherein the antibody is hLept-1, hLept-2, hLept-3, hLept-4, hLept-5, or hLept-6, and wherein the specific binding fragment is obtained from hLept-1, hLept-2, hLept-3, hLept-4, hLept-5, or hLept-6.

3. The method of claim 1, wherein the antibody or specific binding fragment has a variable heavy chain (V_{H}) CDR1 sequence as set forth in SEQ ID NOs: 1, 2, 3, 4 or 5; a V_{H} CDR2 sequence as set forth in SEQ ID NOs: 6, 7, 8, 9 or 10; a V_{H} CDR3 sequence as set forth in SEQ ID NOs: 11, 12, 13, 14 or 15; a variable light chain (V_{L}) CDR1 sequence as set forth in SEQ ID NOs: 16, 17, 18, 19 or 20; a V_{L} CDR2 sequence as set forth in SEQ ID NOs: 21, 22, 23, 24 or 25; and a V_{L} CDR3 sequence as set forth in SEQ ID NOs: 26, 27, 28, 29 or 30.

4. The method of claim 1, wherein the gene editing composition comprises at least one polynucleotide encoding an RNA-guided DNA endonuclease protein or an RNA-guided DNA endonuclease protein, and at least one guide RNA (gRNA) having a spacer sequence complementary to a leptin polynucleotide sequence.

5. The method of claim 1, wherein the leptin antagonist is a leptin mutein.

6. The method of claim 5, wherein the leptin mutein is LanI (L39A/D40A/F41A mutant), Lan2 (L39A/D40A/F41A/I42A mutant), or SHLA (D23L/L39 A/D40A/F41A mutant.

7. The method of claim 1, wherein an amount of circulating leptin is lowered by 30 to 90% in the subject.

8. A method of inducing weight loss in a patient in need thereof comprising administering a treatment regimen comprising a therapeutic agent for lowering circulating leptin and a GLP-1 agonist to a subject in need thereof, wherein the GLP-1 agonist is liraglutide, exenatide, albiglutide, dulaglutide, lixisenatide, or semaglutide, and
wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

9. The method of claim 8, further comprising removing the GLP-1 agonist from the treatment regimen after a desired weight level is achieved.

10. The method of claim 8, wherein the antibody is hLept-1, hLept-2, hLept-3, hLept-4, hLept-5, or hLept-6, and wherein the specific binding fragment is obtained from hLept-1, hLept-2, hLept-3, hLept-4, hLept-5, or hLept-6.

11. The method of claim 8, wherein the antibody or specific binding fragment has a variable heavy chain (V_{H}) CDR1 sequence as set forth in SEQ ID NOs: 1, 2, 3, 4 or 5; a V_{H} CDR2 sequence as set forth in SEQ ID NOs: 6, 7, 8, 9 or 10; a V_{H} CDR3 sequence as set forth in SEQ ID NOs: 11, 12, 13, 14 or 15; a variable light chain (V_{L}) CDR1 sequence as set forth in SEQ ID NOs: 16, 17, 18, 19 or 20; a V_{L} CDR2 sequence as set forth in SEQ ID NOs: 21, 22, 23, 24 or 25; and a V_{L} CDR3 sequence as set forth in SEQ ID NOs: 26, 27, 28, 29 or 30.

12. The method of claim 8, wherein the gene editing composition comprises at least one polynucleotide encoding an RNA-guided DNA endonuclease protein or an RNA-guided DNA endonuclease protein, and at least one guide RNA (gRNA) having a spacer sequence complementary to a leptin polynucleotide sequence.

13. The method of claim 8, wherein the leptin antagonist is a leptin mutein.

14. The method of claim 13, wherein the leptin mutein is LanI (L39A/D40A/F41A mutant), Lan2 (L39A/D40A/F41A/I42A mutant), or SHLA (D23L/L39 A/D40A/F41A mutant.

15. A method of reducing weight gain resulting from administration of an anti-psychotic medication comprising administering an anti-psychotic medication and a therapeutic agent for lowering circulating leptin to a subject in need thereof,
wherein the therapeutic agent is an antibody or specific binding fragment thereof, a leptin antagonist, a leptin targeting antisense oligonucleotide, a leptin targeting small interfering RNA (siRNA), a leptin targeting short hairpin RNA (shRNA), or a gene editing composition directed to at least one target sequence of a leptin polynucleotide.

16. The method of claim 15, wherein the antibody is hLept-1, hLept-2, hLept-3, hLept-4, hLept-5, or hLept-6, and wherein the specific binding fragment is obtained from hLept-1, hLept-2, hLept-3, hLept-4, hLept-5, or hLept-6.

17. The method of claim 15, wherein the antibody or specific binding fragment has a variable heavy chain (V_{H}) CDR1 sequence as set forth in SEQ ID NOs: 1, 2, 3, 4 or 5; a V_{H} CDR2 sequence as set forth in SEQ ID NOs: 6, 7, 8, 9 or 10; a V_{H} CDR3 sequence as set forth in SEQ ID NOs: 11, 12, 13, 14 or 15; a variable light chain (V_{L}) CDR1 sequence as set forth in SEQ ID NOs: 16, 17, 18, 19 or 20; a V_{L} CDR2 sequence as set forth in SEQ ID NOs: 21, 22, 23, 24 or 25; and a V_{L} CDR3 sequence as set forth in SEQ ID NOs: 26, 27, 28, 29 or 30.

18. The method of claim 15, wherein the gene editing composition comprises at least one polynucleotide encoding an RNA-guided DNA endonuclease protein or an RNA-guided DNA endonuclease protein, and at least one guide RNA (gRNA) having a spacer sequence complementary to a leptin polynucleotide sequence.

19. The method of claim 15, wherein the leptin antagonist is a leptin mutein.

20. The method of claim 19, wherein the leptin mutein is LanI (L39A/D40A/F41A mutant), Lan2 (L39A/D40A/F41A/I42A mutant), or SHLA (D23L/L39 A/D40A/F41A mutant.
